(19) United States
(12) Patent Application Publication
(10) Pub. No.: US 2014/0194429 A1
(43) Pub. Date: Jul. 10, 2014

(54) COMPOSITIONS FOR TREATMENT OF CANCER

(71) Applicant: NovaRx Corporation, San Diego, CA (US)

(72) Inventors: Feridoun KARIMI-BUSHERI, Edmonton (CA); Habib FAKHRAI, La Jolla, CA (US)

(21) Appl. No.: 13/801,876

(22) Filed: Mar. 13, 2013

Related U.S. Application Data
(60) Provisional application No. 61/749,219, filed on Jan. 4, 2013.

(51) Int. Cl. C07D 471/04; C07D 257/06; C07D 215/24; C07D 251/46

(52) U.S. Cl. CPC C07D 471/04; C07D 251/46; C07D 257/06; C07D 215/24
USPC 514/245; 544/194; 514/292; 546/81; 514/381; 548/253; 514/311; 546/174

(57) ABSTRACT

Compounds that are specifically toxic to cancer stem cells are disclosed.

### CROSS-REFERENCE TO RELATED APPLICATION

This application claims priority from U.S. provisional application 61/749,219 filed 4 Jan. 2013. The contents of this document are incorporated herein by reference.

### TECHNICAL FIELD

The invention is in the field of pharmaceuticals useful in cancer treatment. More particularly, it relates to small molecules that are particularly toxic to cancer stem cells.

### BACKGROUND ART

It has been recognized for some time that the cells contained in a particular cancer are heterogeneous and have different susceptibilities to chemotherapeutic treatments as well as radiation therapy. In particular, many cancers contain cancer stem cells that are chiefly responsible for the initiation and spread of the cancer, including metastases. Cancer stem cells are resistant to conventional therapy and it is particularly important to eradicate these cells to prevent progression and metastasis of cancer.

Karimi-Busheri, F., et al., J. Stem Cells (2011) 6:9-20 reported the isolation of stem cells both from non-small cell lung tumor specimens and from lung tumor cell lines and provide a system for identification of such stem cells using CD38 in combination with CD24 as biomarkers and a combination of these biomarkers with overexpression of ALDH1 and EpCAM. These markers provide a signature for tumor-initiating cells in the H-460 lung cancer cell line.

It has been reported by Bernstein, N. K., et al., Anticancer Agents in Med. Chem. (2008) 8:358-367 that a polynucleotide kinase which is essential for DNA repair is critical to the resistance of cancer cells, including cancer stem cells, to DNA-damaging agents. This paper suggests that inhibitors of polynucleotide kinase would be effective in overcoming the resistance of these cells to radiation therapy and to other forms of chemotherapeutic treatment.

PCT publication No. WO2010/139069 assigned to the University of Alberta discloses a series of 6,7a-dihydro-1H-pyrrolo[3,4-b]pyridine-5,7(2H,4aH) diones as inhibitors of this enzyme. This work was also reported by the same group in an article by Freschauf, G. K., et al., Cancer Res. (2009) 69:7739-7746.

There remains a need for additional small molecule pharmaceuticals that will provide effective treatment for cancer cells, including specifically cancer stem cells and that will overcome any resistance of these cells to additional chemotherapeutic agents.

### SUMMARY OF THE INVENTION

The present invention is related to pharmaceutical compositions that have been demonstrated to have enhanced ability to effect cell death and DNA destruction in cancer stem cells even in comparison to their capacity to do so in other cancer cells.

Accordingly, in one aspect, the invention is directed to pharmaceutical compositions comprising an active ingredient selected from the group consisting of

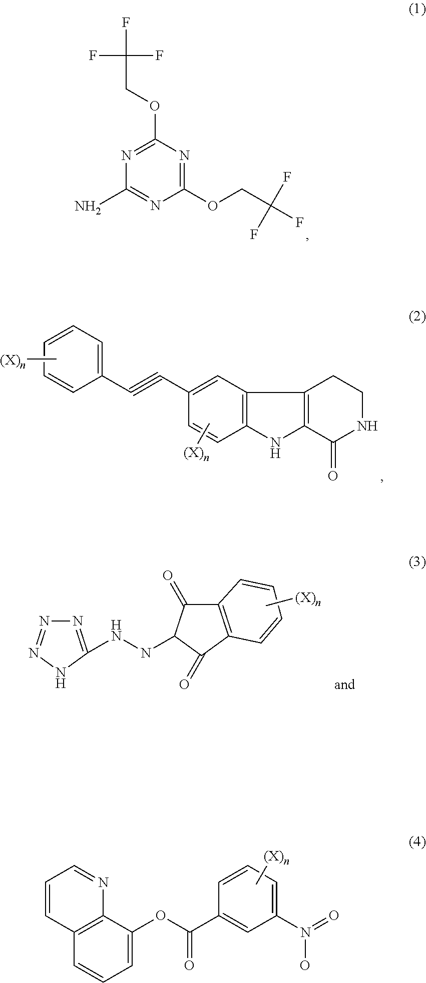

wherein each X is independently selected from the group consisting of halo, nitro, OR², OCOR², COOR², R²NCOR², CONR²₂, COR², NR²₂, S(O)ₘR², —CN and R³,

where each R² is independently H or alkyl (1-4)

R³ is alkyl (1-6) or substituted alkyl (1-6) wherein the substituents are halo, OR² or NR²₂,

m is 0, 1, or 2, and

n is 0, 1, 2 or 3.

In another aspect, the invention is directed to pharmaceutical compositions comprising as an active ingredient a compound of formula (A):

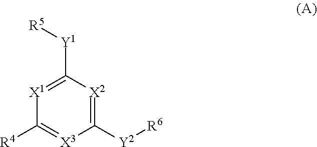

wherein
R⁴ is halo, nitro, OR⁷, OCOR⁷, COOR⁷, R⁷NCOR⁷, CONR⁷₂, COR⁷, NR⁷₂, S(O)ₘR⁷, —CN or R⁸,
R⁵ and R⁶ are each independently alkyl or substituted alkyl wherein the substituents are independently halo, OR⁷, or NR⁷₂,
X¹, X², and X³ are each independently N or CR⁷, wherein at least one of X¹, X², and X³ is N,
Y¹ and Y² are each independently CH₂, O, S, or NH, where each R⁷ is independently H or alkyl,
R⁸ is alkyl or substituted alkyl wherein the substituents are independently halo, OR⁷ or NR⁷₂, and
m is 0, 1, or 2;

or a pharmaceutically acceptable salt thereof.

In another aspect, the invention is directed to pharmaceutical compositions comprising as an active ingredient a compound of formula (B):

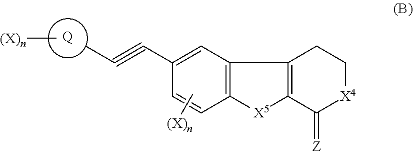

wherein
X⁴ is O, NH, or CH₂,
X⁵ is O, S, NH, or CH₂,
Z is O, S, or NH,
Q is aryl, heteroaryl, cycloalkyl, or heterocycloalkyl,
each X is independently selected from the group consisting of halo, nitro, OR², OCOR², COOR², R²NCOR², CONR²₂, COR², NR²₂, S(O)ₘR², —CN and R³,
where each R² is independently H or alkyl,
R³ is alkyl or substituted alkyl wherein the substituents are independently halo, OR² or NR²₂,
m is 0, 1, or 2, and
each n is independently 0, 1, 2 or 3;

or a pharmaceutically acceptable salt thereof.

In other aspects, the invention is directed to methods to treat cancers in subjects using the invention pharmaceutical compositions or active ingredients thereof.

In other aspects, the invention is directed to kits comprising pharmaceutical compositions or active ingredients thereof as described herein and instructions for their use in the treatment of cancers.

In other aspects, the invention is directed to articles of manufacture comprising pharmaceutical compositions or active ingredients thereof as described herein for treatment of cancers.

### BRIEF DESCRIPTION OF THE DRAWINGS

FIG. 1 shows typical results of an assessment of the effect of the compounds of the invention on the viability of adherent cells vs. stem cells derived from the H460 cell line.

### DETAILED DESCRIPTION

The disclosures of the publications cited in this specification, including patents, are herein incorporated by reference.

As used herein, the terms “including,” “containing,” and “comprising” are used in their open, non-limiting sense.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

The “alkyl” groups in the compounds described herein may be straight or branched chain or cyclic and include, for example, methyl, ethyl, tertiary butyl, cyclopentyl, and groups that, in light of the ordinary skill in the art and the teachings provided herein, would be considered equivalent to any one of the foregoing examples. The alkyl group may have from 1 to 12 carbon atoms in the chain. Particular alkyl groups are those having 1 to 8 carbon atoms, 1 to 6 carbon atoms, or 1 to 4 carbon atoms.

The term “aryl” refers to a monocyclic, fused bicyclic, or fused polycyclic aromatic ring having from 3 to 12 carbon atoms. Particular aryl groups are those having from 3 to 8 carbon atoms or from 5 to 7 carbon atoms. Illustrative examples of aryl groups include phenyl, naphthalene, anthracene, and groups that, in light of the ordinary skill in the art and the teachings provided herein, would be considered equivalent to any one of the foregoing examples.

The term “cycloalkyl” refers to a monocyclic, or fused, bridged, or spiro polycyclic ring structure that is saturated or partially saturated and has from 3 to 12 carbon atoms. Particular heterocycloalkyl groups are those having from 3 to 8 carbon atoms or from 5 to 7 carbon atoms. Illustrative examples of cycloalkyl groups include cyclopropane, cyclopropene, cyclobutane, cyclobutene, cyclopentane, cyclopentadiene, cyclohexane, cyclohexene, cyclohexadiene, cycloheptane, cyclooctane, adamantine, and groups that, in light of the ordinary skill in the art and the teachings provided herein, would be considered equivalent to any one of the foregoing examples.

The term “heteroaryl” refers to a monocyclic, fused bicyclic, or fused polycyclic aromatic heterocycle (ring structure having ring atoms selected from carbon atoms and up to four heteroatoms selected from nitrogen, oxygen, and sulfur) having from 3 to 12 ring atoms per heterocycle. Particular heteroaryl groups are those having from 3 to 8 ring atoms or from 5 to 7 ring atoms per ring structure. Illustrative examples of heteroaryl groups include the following entities, in the form of properly bonded moieties:

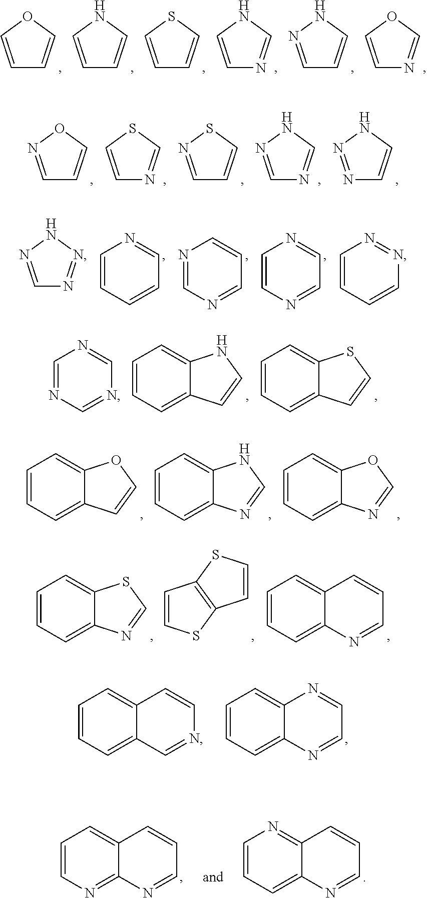

The term “heterocycloalkyl” refers to a monocyclic, or fused, bridged, or spiro polycyclic ring structure that is saturated or partially saturated and has from 3 to 12 ring atoms per ring structure selected from carbon atoms and up to three heteroatoms selected from nitrogen, oxygen, and sulfur. Particular heterocycloalkyl groups are those having from 3 to 8 ring atoms or from 5 to 7 ring atoms per ring structure. The ring structure may optionally contain up to two oxo groups on carbon or sulfur ring members. Illustrative entities, in the form of properly bonded moieties, include:

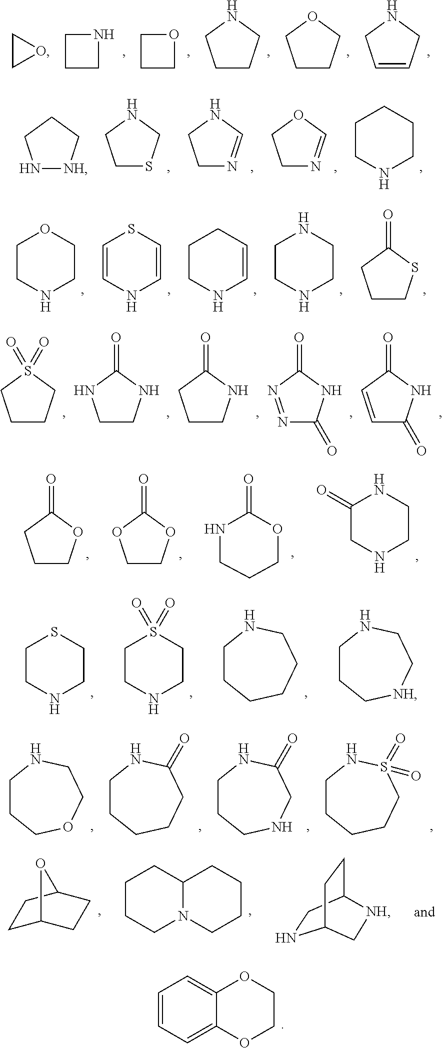

The term “halogen” represents chlorine, fluorine, bromine, or iodine. The term “halo” represents chloro, fluoro, bromo, or iodo.

Those skilled in the art will recognize that the species of aryl, cycloalkyl, heteroaryl and heterocycloalkyl groups listed or illustrated above are not exhaustive, and that additional species within the scope of these defined terms may also be selected.

The term “substituted” means that the specified group or moiety bears one or more substituents. The term “unsubstituted” means that the specified group bears no substituents. Where the term “substituted” is used to describe a structural system, the substitution is meant to occur at any valency-allowed position on the system.

The terms “alkyl (i-j),” “aryl (i-j),” and “cycloalkyl (i-j),” where j>i, refer, respectively, to alkyl, aryl and cycloalkyl groups containing a minimum of i number of carbon atoms and a maximum of j number of carbon atoms and are inclusive of all numbers of carbon atoms from i through j. The terms “heteroaryl (i-j)” and “heterocycloalkyl (i-j), wherein j>i, refer, respectively, to heteroaryl and heterocycloalkyl groups containing a minimum of i number of ring atoms and a maximum of j number of ring atoms, wherein the ring atoms include both carbon and heteroatoms in the ring, and are inclusive of all numbers of ring atoms from i through j.

Any formula given herein is intended to represent compounds having structures depicted by the structural formula as well as certain variations or forms. In particular, compounds of any formula given herein may have asymmetric centers and therefore exist in different stereoisomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers. Additionally, any formula given herein is intended to refer also to any one of hydrates, solvates, and amorphous and polymorphic forms of such compounds, and mixtures thereof, even if such forms are not listed explicitly. In some embodiments, the solvent is water and the solvates are hydrates.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds described herein include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labeled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds described herein and prodrugs thereof can generally be prepared by carrying out the procedures disclosed in the schemes or in the examples and preparations described below by substituting a readily available isotopically labeled reagent for a non-isotopically labeled reagent.

In one aspect, the invention is directed to pharmaceutical compositions comprising as an active ingredient a compound of formula (A), as described herein.

In some embodiments, each of X¹, X², and X³ is N. In other embodiments, X¹ and X³ are N and X² is CH₂. In some embodiments, R⁴ is halo, nitro, OR⁷, or NR⁷₂. In some embodiments, R⁴ is NR⁷₂, for example, NH₂, NHCH₃, or N(CH₃)₂. In other embodiments, R⁴ is OR⁷, for example, OH or OCH₃. In a particular embodiment, R⁴ is NH₂. In some embodiments, each of X¹, X², and X³ is N, and R⁴ is NH₂.

In some embodiments, each R⁷ is independently H or alkyl (1-4). In other embodiments, each R⁷ is independently H or alkyl (1-2). In some embodiments, R⁸ is alkyl (1-6) or substituted alkyl (1-6) wherein the substituents are halo, OR⁷, or NR⁷₂. In some embodiments, R⁸ is alkyl (1-4). In other embodiments, R⁸ is substituted alkyl (1-4) wherein the substituents are halo, OH, OCH₃, or NH₂.

Y¹ and Y² may be the same or different. In some embodiments, Y¹ and Y² are both O. In other embodiments, Y¹ and Y² are both NH. In yet other embodiments, Y¹ and Y² are both CH₂. In some embodiments, R⁵ and R⁶ are each independently alkyl substituted with one or more halo. In some embodiments, R⁵ and R⁶ are each independently alkyl (1-4) substituted with one or more halo, OH, OCH₃, or NH₂. In some embodiments, R⁵ and R⁶ are each alkyl (1-2) independently substituted with one or more halo. In a particular embodiment, R⁵ and R⁶ are both CH₂CF₃. In some embodiments, Y¹ and Y² are both 0, and R⁵ and R⁶ are each independently alkyl (1-4) substituted with one or more halo. In a particular embodiment, Y¹ and Y² are both 0, and R⁵ and R⁶ are both CH₂CF₃.

In one embodiment, the compound of formula (A) is a compound of formula (1).

In one aspect, the invention is directed to pharmaceutical compositions comprising as an active ingredient a compound of formula (B), as described herein.

In some embodiments, X⁴ is NH. In other embodiments, X⁴ is O. In some embodiments, X⁵ is NH. In other embodiments, X⁵ is O. In yet other embodiments, X⁵ is CH₂. In some embodiments, Z is O. In some embodiments, Z is S. In yet other embodiments, Z is NH. In a particular embodiment, X⁴ is NH, X⁵ is NH, and Z is O.

In some embodiments, Q is aryl (3-8), heteroaryl (3-8), cycloalkyl (3-8), or heterocycloalkyl (3-8). In some embodiments, Q is aryl or heteroaryl. In some embodiments, Q is aryl. In a particular embodiment, Q is phenyl. In some embodiments, Q is heteroaryl. In some embodiments, Q is pyridyl, pyrimidinyl, or pyrazinyl. In some embodiments, Q is phenyl, and each n is 0.

In some embodiments, each X is independently halo, OR², or NR²₂. In some embodiments, each X is independently halo, OH, OCH₃, or NH₂. In some embodiments, R² is independently H or alkyl (1-4). In other embodiments, R² is independently H or alkyl (1-2). In some embodiments, R³ is alkyl (1-6) or substituted alkyl (1-6) wherein the substituents are halo, OR², or NR²₂, and R² is independently H or alkyl (1-4). In some embodiments, R³ is alkyl (1-2) or substituted alkyl (1-2) wherein the substituents are halo, OH, OCH₃, or NH₂.

In one embodiment, the compound of formula (B) is a compound of formula (2).

In one aspect, the pharmaceutical compositions of the invention contain as at least one active ingredient a compound described herein.

Pharmaceutically acceptable salts include salts of a free acid or base of a compound represented herein that is non-toxic, biologically tolerable, or otherwise biologically suitable for administration to the subject. See, generally, Berge, S. M., et al. “Pharmaceutical Salts,” J. Pharm. Sci. (1977) 66:1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, or both types of functional groups, and accordingly react with a number of inorganic or organic bases, and inorganic and organic acids, to form a pharmaceutically acceptable salt. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates.

For treatment purposes, pharmaceutical compositions comprising compounds described herein may further comprise one or more pharmaceutically-acceptable excipients. A pharmaceutically-acceptable excipient is a substance that is non-toxic and otherwise biologically suitable for administration to a subject. Such excipients facilitate formulation and administration of a compound described herein and are compatible with the active ingredient. Examples of pharmaceutically-acceptable excipients include stabilizers, lubricants, surfactants, diluents, anti-oxidants, binders, coloring agents, emulsifiers, or taste-modifying agents. In preferred embodiments, pharmaceutical compositions are sterile compositions.

The pharmaceutical compositions described herein may be formulated as solutions, emulsions, suspensions, or dispersions in suitable pharmaceutical solvents or carriers, or as pills, tablets, lozenges, suppositories, powders for reconstitution, or capsules along with solid carriers according to conventional methods known in the art for preparation of various dosage forms. For topical applications, the compounds described herein are preferably formulated as creams or ointments or a similar vehicle suitable for topical administration.

For treatment of subjects using the pharmaceutical compositions of the invention or the compounds described herein per se, a variety of protocols and methods of administration may be employed depending on the nature of the subject, the particular kind of tumor, the judgment of the practitioner and the stage of cancer. Various methods of administration are known in the art including parenteral administration, oral or other digestive system-based administration, transmucosal, transdermal administration or administration by suppository. Parenteral administration may include IP, IV and subcutaneous forms.

The term “treat” or “treating” as used herein is intended to refer to administration of a compound described herein to a subject for the purpose of creating a therapeutic benefit. Treating includes reversing, ameliorating, alleviating, inhibiting the progress of, or lessening the severity of, a disease, disorder, or condition, or one or more symptoms of cancer. The term “subject” refers to a patient in need of such treatment. The subjects in important aspects of the invention are human subjects, but treatment is not limited to them. Treatment for malignancy is also important in other vertebrate species including various forms of livestock such as cows, pigs, sheep and goats as well as companion animals, fish and birds. In particular, the compounds described herein may be used to treat laboratory animal tumor models, such as rats, mice, rabbits and the like in order to optimize dosage regimens and protocols.

In treatment methods provided herein, “an effective amount” means an amount or dose sufficient to generally bring about the desired therapeutic benefit in subjects needing such treatment. Effective amounts or doses of the compounds described herein may be ascertained by routine methods, such as modeling, dose escalation or clinical trials, taking into account routine factors, e.g., the mode or route of administration or drug delivery, the pharmacokinetics of the agent, the severity and course of the infection, the subject's health status, condition, and weight, and the judgment of the treating physician. An exemplary dose is in the range of about 1 ug to 2 mg of active compound per kilogram of subject's body weight per day, preferably about 0.05 to 100 mg/kg/day, or about 1 to 35 mg/kg/day, or about 0.1 to 10 mg/kg/day. The total dosage may be given in single or divided dosage units (e.g., BID, TID, QID).

As noted above, the compounds described herein are particularly effective in decreasing the viability of cancer stem cells in addition to their ability to effect cell death in non-stem cell forms of cancer—i.e., adherent cells. Accordingly, the pharmaceutical compositions of the invention or the compounds described herein per se are useful in treating subjects harboring malignant tumors.

The mode of administration will also depend on the nature of the formulation employed. Suitable formulations may be found in Remington's Pharmaceutical Sciences, latest edition, Mack Publishing Co., Easton, Pa. The compositions may be simple formulations with conventional excipients or may include liposomes, micelles, controlled release systems or other polymeric supports and may include other active ingredients unrelated to the compounds described herein. For solid tumors, it is also possible to provide the compounds described herein directly by intratumoral administration. Dosage levels are variable depending on the practitioner's judgment and the nature of the subject but can readily be determined from the behavior of the compounds in animal models commonly used to optimize dosages.

Methods for synthesizing the compounds described herein are well known in the art and representative members of this genus are commercially available. Illustrative synthetic methods for the general preparation of the compounds described herein are presented below. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Furthermore, one of skill in the art will recognize that the transformations shown in the schemes below may be performed in any order that is compatible with the functionality of the particular pendant groups. Each of the reactions depicted in the general schemes is preferably run at a temperature from about 0° C. to the reflux temperature of the organic solvent used. Unless otherwise specified, the variables are as defined above in reference to formulas (A) and (B).

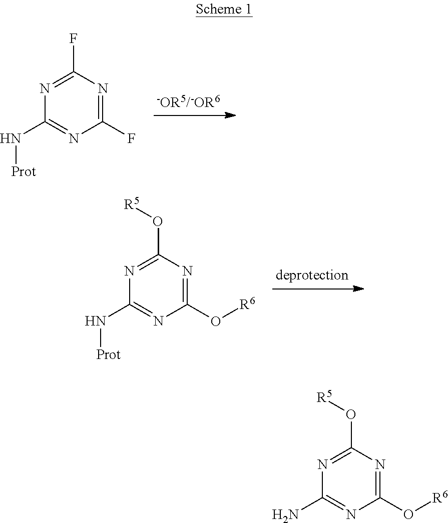

Referring to Scheme 1, certain compounds of formula (A) can be synthesized from an appropriate halo-substituted heteroaryl compound. “Prot” refers to a protecting group, which may be any protecting group that is stable in the reaction conditions used. The starting material may be subject to nucleophilic aromatic substitution with an appropriate alkoxide, followed by removal of any protecting groups used during the synthesis. If R⁵ and R⁶ are different, separation methods such as silica gel chromatography or HPLC can be used to separate the mixture of products. Other compounds of formula (A) can be synthesized using methods analogous to Scheme 1.

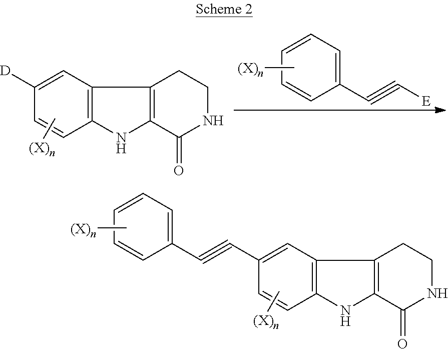

Referring to Scheme 2, certain compounds of formula (B) can be synthesized via coupling of a tetrahydryo-1H-pyrido[3,4-b]indol-1-one with a propynyl benzene under appropriate conditions, where D and E are appropriate functional groups for carrying out the coupling reaction. Appropriate protecting groups can be used as described herein.

The following examples are intended to illustrate but not to limit the invention.

### Example 1

### Isolation of Stem Cells

Stem cells were isolated from non-small cell lung tumor specimens of human tumors developed in NOD/SCID mice according to the description set forth in Karimi-Busheri, F., et al., J. Stem Cells (2011) supra, incorporated herein by reference. Briefly, a combination of three criteria were used:

Formation of a subpopulation of cells identifiable by efflux of Hoechst 33342 by ABC transporters via flow cytometry analysis;

Formation of floating spheres in culture; and

Expression of the markers CD133, and CD24/CD38 ratio. These enriched populations of stem cells were used in the experiments below.

### Example 2

### Viability Assay

The compounds described herein showed activity in a cell viability assay conducted as follows:

Samples of 5×10³ cells per well were seeded onto 96-well plates along with the desired concentration of compound of formula (1) dissolved in DMSO. Control wells contained cells without treatment and medium without cells was used as a background control. The plates were incubated for 120 hours at 37° C. and then assayed for viability using the commercially available PrestoBlue® Cell Viability Staining assay marketed by Invitrogen, Carlsbad, Calif. After incubation, the plates were irradiated at 540-570 nm and the emission at 590 nm measured. Cell death was shown by a decrease in fluorescence.

The compounds described herein are able to decrease viability by at least 40% in this assay when the concentration of compound is 100 μM.

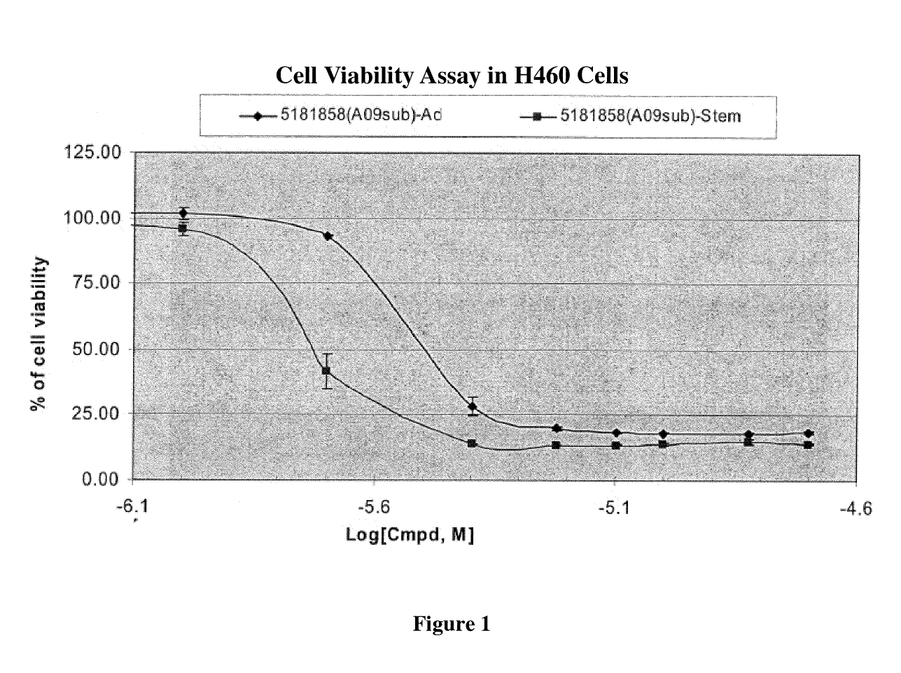

Varying concentrations of the compounds described herein were used to determine IC₅₀. The compounds described herein have IC₅₀'s between 1 and 5 μM. A typical result is shown in FIG. 1. As shown, the efficacy of the compound is greater on stem cells than in adherent cancer cells.

### Example 3

### Determination of Maximum Tolerated Dose

For each compound tested in this assay, 10 female BALB/c nu/nu mice were used in an in vivo study to determine maximum tolerated dose (MTD). An MTD of 25 μM was found for these compounds.

### Example 4

### In Vivo Efficacy Model

NOD/SCID mice containing human tumor stem cells of a non-small cell lung tumor specimen are used to evaluate dosage levels for the compounds described herein.

### Example 5

### Exemplary Compounds

Compounds typical of those of formula (1), (2), (3), (4), (A), or (B) are those wherein each n is 0.

1. A pharmaceutical composition comprising as an active ingredient a compound of formula (A): 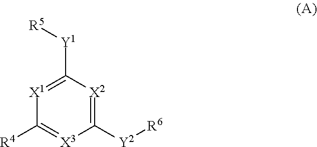
wherein
R⁴ is halo, nitro, OR⁷, OCOR⁷, COOR⁷, R⁷NCOR⁷, CONR⁷₂, COR⁷, NR⁷₂, S(O)ₘR⁷, —CN or R⁸,
R⁵ and R⁶ are each independently alkyl or substituted alkyl wherein the substituents are independently halo, OR⁷, or NR⁷₂,
X¹, X², and X³ are each independently N or CR⁷, wherein at least one of X¹, X², and X³ is N,
Y¹ and Y² are each independently CH₂, O, S, or NH,
where each R⁷ is independently H or alkyl,
R⁸ is alkyl or substituted alkyl wherein the substituents are independently halo, OR⁷ or NR⁷₂, and
m is 0, 1, or 2;
or a pharmaceutically acceptable salt or solvate thereof.

2. The pharmaceutical composition of claim 1, wherein R⁴ is NH₂.

3. The pharmaceutical composition of claim 1, wherein Y¹ and Y² are each O, and R⁵ and R⁶ are each CH₂CF₃.

4. The pharmaceutical composition of claim 1, wherein X¹, X² and X³ are each N.

5. A pharmaceutical composition comprising as an active ingredient a compound of formula (B): 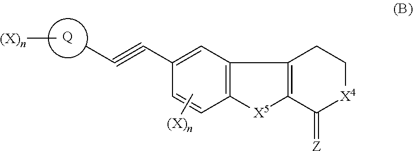
wherein
X⁴ is O, NH, or CH₂,
X⁵ is O, S, NH, or CH₂,
Z is O, S, or NH,
Q is aryl, heteroaryl, cycloalkyl, or heterocycloalkyl,
each X is independently selected from the group consisting of halo, nitro, OR², OCOR², COOR², R²NCOR², CONR²₂, COR², NR²₂, S(O)ₘR², —CN and R³,
where each R² is independently H or alkyl,
R³ is alkyl or substituted alkyl wherein the substituents are independently halo, OR² or NR²₂,
m is 0, 1, or 2, and
each n is independently 0, 1, 2 or 3;
or a pharmaceutically acceptable salt thereof.

6. The pharmaceutical composition of claim 5, wherein X⁴ is NH.

7. The pharmaceutical composition of claim 5, wherein X⁵ is NH.

8. The pharmaceutical composition of claim 5, wherein Z is O.

9. The pharmaceutical composition of claim 5, wherein Q is phenyl.

10. The pharmaceutical composition of claim 5, wherein each n is 0.

11. A pharmaceutical composition comprising an active ingredient selected from the group consisting of 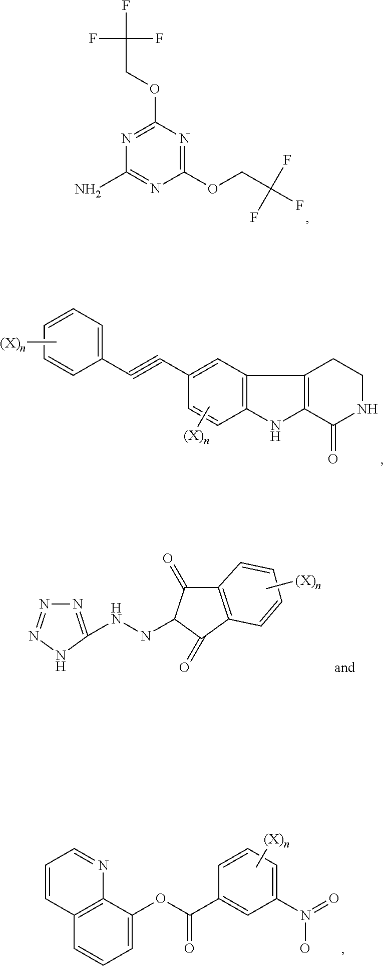
wherein each X is independently selected from the group consisting of halo, nitro, OR², OCOR², COOR², R²NCOR², CONR²₂, COR², NR²₂, S(O)ₘR², —CN and R³,
where each R² is independently H or alkyl (1-4)
R³ is alkyl (1-6) or substituted alkyl (1-6) wherein the substituents are halo, OR² or NR²₂, and
n is 0, 1, 2 or 3.

12. The composition of claim 11 wherein each n is O.

13. A method to treat cancer in a subject in need thereof which comprises administering to said subject an effective amount of the composition of claim 1.

14. A method to treat cancer in a subject in need thereof which comprises administering to said subject an effective amount of the composition of claim 5.

15. A method to treat cancer in a subject in need thereof which comprises administering to said subject an effective amount of the composition of claim 11.
